(19) United States
(12) Patent Application Publication
(10) Pub. No.: US 2022/0146539 A1
(43) Pub. Date: May 12, 2022

(54) CONSUMER OPERATED ANALYSIS OF CANNABIS BASED PRODUCTS AND OTHER PRODUCTS, RELATED DATA PROCESSING, SYSTEMS AND METHODS THEREOF

(71) Applicant: QUANA-WAYS LTD., Herzeliya (IL)

(72) Inventors: Zohar KEREM, Rehovot (IL); Adi NAALI, Kfar Vitkin (IL); Ravid NAALI, Kanaf (IL)

(21) Appl. No.: 17/433,849

(22) PCT Filed: Feb. 26, 2020
(86) PCT No.: PCT/IL2020/050219

§ 371 Date: Aug. 25, 2021

Related U.S. Application Data
(60) Provisional application No. 62/810,434, filed on Feb. 26, 2019.

(51) Int. Cl. G01N 33/94

(52) U.S. Cl. CPC G01N 33/948; G01N 33/946

(57) ABSTRACT

A system for analyzing a Cannabis product, comprising: (1) at least one detecting unit; the unit comprising (i) at least one detector; and (ii) at least one detection/sensor/sensing source; (iii) a computer readable medium (CRM), configured to process the detection results; (2) a processing unit, configured to read the detection results; (3) a communication unit, configured to transmit the results; (4) an analysis database; and (5) an analysis unit, configured to analyses the product results and compare the results to the database; wherein the system provides a report, disclosing the quality of the sample.

### FIELD OF THE INVENTION

The invention is in the field of point-of-care analysis of Cannabis, Cannabis-based products and other products.

### BACKROUND OF THE INVENTION

Medicinal Cannabis is a broad term for a variety of Cannabis-based medicines that are widely used for the management and treatment of a number of disorders. Cannabis has been show to help patents of epilepsy and Parkinson disorder among many other disorders. Cannabis also has potential to help patent suffering from chronic pain and cancer by managing symptoms or the side effects of other treatments, such as chemotherapy.

Cannabinoids are a diverse class of chemical compounds that act on the cannabinoid receptors. Phyto-cannabinoids are cannabinoids found in plants, mainly found in the Cannabis genus. Over 110 cannabinoids have been identified in Cannabis. The different cannabinoids exhibit different pharmacological and chemical characteristics. The ration of the different cannabinoids in a product can affect the potential use of medicinal Cannabis are its therapeutic target.

US application 20170059536A1 describes a Cannabis analysis system including a solvent input port configured to receive a flow of a solvent, a sample input port configured to receive a chemical mixture including a sample, a pump configured to move the solvent and chemical mixture, an extraction cartridge coupled to the pump and configured to separate a combination of the chemical mixture and the flow of the solvent into two or more partially-separated chemical components, a spectroscopy cell configured to generate a chromatogram including two or more overlapping absorption peaks corresponding to the two or more partially-separated chemical components, and a controller configured to determine a chemical concentration of each partially-separated chemical component of the two or more partially-separated chemical components.

US application 201701991681A1 describes systems and methods of evaluating Cannabis and cannabinoid products for public safety, quality control and quality assurance purposes for research and public use purposes. Embodiments of the invention facilitate suppliers and consumers of a Cannabis and/or cannabinoid product to evaluate the origin, efficacy, potency, and quality of the product. Embodiments of the invention also include Cannabis processing center where samples of the products are analyzed for research studies to test and set parameters for third parties. Embodiments of the invention include, for example, analyzing Cannabis products to determine quality and quantity of desired components and undesired components, determining concentrations of cannabinoids in the product, and comparing measures of components in the product against regulations. Embodiments of the invention further include, for example, an online testing facility for determine whether Cannabis products meet a plurality of regulations or guidelines.

There exists a long felt need for a point-of-care method for the analysis of a Cannabis product for patents.

### SUMMARY

A system for analyzing a Cannabis product 10, comprising:
a. at least one detecting unit 11 22; the unit comprising
   i. at least one detector 92; and
   ii. at least one detection/sensor/sensing source;
   iii. a computer readable medium (CRM) , the CRM configured to process the detection results;
b. a processing unit 12 23, the unit configured to read the detection results;
c. a communication unit 13 24, the unit configured to transmit the results;
d. an analysis database 15 26; and
an analysis unit 14 25, the unit configured to analyses the product results and compare the results to the database;
wherein the system provides a report, the report disclosing the quality of the sample.

It is another object of the current invention to disclose the system as defined above, wherein the detector 11 22 is selected from a group of technologies consisting of chemical detectors, spectroscopic detectors, electrochemical, luminescent, fluorescent, optical, polymer, hydrogels, surface acoustic wave (SAW) electronic-nose sensors, as immunological (antibody), enzyme-linked immunosorbent assay (ELISA), enzymatic biosensor microbial biosensors, fiber optic biosensors, quartz crystal microbalance (QCM) and electromagnetic QCM (EQCM). It is another object of the current invention to disclose the system as defined in any of the above, wherein the detector is configured to detect at least one environmental condition.

It is another object of the current invention to disclose the system as defined in any of the above, wherein the conditions are selected from a group consisting of temperate and humidity.

It is another object of the current invention to disclose the system as defined in any of the above, wherein the detector is configured to analyze volatile compounds.

It is another object of the current invention to disclose the system as defined in any of the above, wherein the system additionally comprises a sample holder.

It is another object of the current invention to disclose the system as defined in any of the above, wherein the holder is configured to position a solid sample or a liquid sample.

It is another object of the current invention to disclose the system as defined in any of the above, wherein the holder additionally comprises a heating unit.

It is another object of the current invention to disclose the system as defined in any of the above, wherein the heating unit is selected from a group consisting of Infrared light, heating coil, electric element.

It is another object of the current invention to disclose the system as defined in any of the above, wherein the communication 13 unit is configured to communicate using wired or wireless technologies selected from a group consisting of, but not limited to, wireless local area networks (WLAN), Low-Power Wide-Area Networks (LPWAN), wireless wide area network (WWAN), wireless sensor networks, Bluetooth, cellular networks, and any combination of.

It is another object of the current invention to disclose the system as defined in any of the above, wherein the database 15 26 comprises at least one of the following:
a. database of medicinal Cannabis detection results/analysis;
b. database of cannabinoid analysis/results;
c. database of impurities detection results/analysis.

It is another object of the current invention to disclose the system as defined in any of the above, wherein the impurities are selected from a group consisting of pesticides, insecticides, toxins, pesticides, fungicides, Biological contaminants (fungi).

It is another object of the current invention to disclose the system as defined in any of the above, wherein the system additional comprises a display for presenting the analysis results.

It is another object of the current invention to disclose the system as defined in any of the above, wherein the system is powered by an internal electricity source, the source being a battery.

It is another object of the current invention to disclose the system as defined in any of the above, wherein the analysis unit 14 25 is configured to communicate the result to another device or system.

It is another object of the current invention to disclose the system as defined in any of the above, wherein the analysis unit is configured to communicate using wired or wireless technologies selected from a group consisting of, but not limited to, wireless local area networks (WLAN), Low-Power Wide-Area Networks (LPWAN), wireless wide area network (WWAN), wireless sensor networks, Bluetooth, cellular networks, and any combination of.

It is another object of the current invention to disclose the system as defined in any of the above, wherein the analysis unit is configured to communicate using computer-mediated communication (CMC), the CMC selected from a group consisting of email, text message, and web-based application.

It is another object of the current invention to disclose the system as defined in any of the above, wherein the communication is by an application installed on a mobile device.

It is another object of the current invention to disclose the system as defined in any of the above, wherein the analysis unit is configured to mine a database for product analysis.

It is another object of the current invention to disclose the system as defined in any of the above, wherein the sample analysis is conducted by a function selected from a group consisting of an agent, a decision tree, an algorithm and any combination thereof.

It is another object of the current invention to disclose the system as defined in any of the above, wherein the agent is configured to analyze the data, the data selected from a group consisting of sample data, detection data and any combination thereof.

It is another object of the current invention to disclose the system as defined in any of the above, wherein the agent is configured to detect the samples tag, classification, sample data, detection data and any combination thereof.

It is another object of the current invention to disclose the system as defined in any of the above, wherein the tag and/or classification is selected from a group consisting of chemical composition,

It is another object of the current invention to disclose the system as defined in any of the above, wherein the analysis is a function determined by employing machine learning.

It is another object of the current invention to disclose the system as defined in any of the above, wherein the system additionally comprises a reporting unit 27, the unit configured to:
a. generate an analysis report;
b. transmit analysis report to a pharmacovigilance unit 28;
c. communicate Cannabis analysis results.

It is another object of the current invention to disclose the system as defined in any of the above, wherein the system additionally comprises a pharmacovigilance unit 28.

It is another object of the current invention to disclose the system as defined in any of the above, wherein the pharmacovigilance unit 28 is configured to:
a. receive health information from the patent;
b. receive health information for a healthcare person;
c. receive health information for a healthcare system;
d. receive Cannabis analysis results from reporting unit;
e. communicate pharmacovigilance data to the reporting unit;

It is another object of the current invention to disclose the system as defined in any of the above, wherein the pharmacovigilance unit is configured to receive health information and to present a questionnaire to the patent or patent care giver, the questionnaire comprising questions regarding patent health, treatment effect, and additional treatments.

It is another object of the current invention to disclose the system as defined in any of the above, wherein the health information comprises long term effects of treatments, the effects including adverse drug reactions, drug-drug interactions, side effects, benefits, effectiveness, and efficacy.

It is another object of the current invention to disclose the system as defined in any of the above, wherein the external person or system is a healthcare person, a patents group or a regulatory body.

It is another object of the current invention to disclose the system as defined in any of the above, wherein the system additionally comprises a product unit 21, configured to input information about the product.

It is another object of the current invention to disclose the system as defined in any of the above, wherein the product information is selected from a group consisting of the product batch, manufacturing date, source, producer, manufacturer, and storage and transport conditions.

It is the object of the current invention to disclose a method analyzing a Cannabis product 100, comprising steps of
a. receiving a Cannabis product 101;
b. obtaining a Cannabis sample 102;
c. providing an detection unit;
d. analyzing the sample with the detection unit 103;
e. processing results 104;
f. transferring results to the analysis unit 105;
g. analyzing results 106;
h. retrieving data from a database 107; and
i. generating a report of the analysis 108;
   wherein the report comprises an analysis of the Cannabis product.

It is another object of the current invention to disclose the method as defined in any of the above, wherein the method additionally comprises a step of heating the sample.

It is another object of the current invention to disclose the method as defined in any of the above, wherein the step of heating the sample is conducted by using a technology selected from a group consisting of Infrared light, heating coil, and electric heater.

It is another object of the current invention to disclose the method as defined in any of the above, wherein the method additionally comprises a step of evaporating volatile compounds of the sample.

It is another object of the current invention to disclose the method as defined in any of the above, wherein the step of detecting the composition of the sample is conducted by a technology selected from a group consisting of chemical detectors, spectroscopic detectors, electrochemical, luminescent, fluorescent, optical, polymer, hydrogels, surface acoustic wave (SAW) electronic-nose sensors, as immunological (antibody), enzyme-linked immunosorbent assay (ELISA), enzymatic biosensor microbial biosensors and fiber optic biosensors

It is another object of the current invention to disclose the method as defined in any of the above, wherein the method additionally comprises a step of retrieving data from a database 107, the database comprising:
a. database of medicinal Cannabis detection results/analysis;
b. database of cannabinoid detection results/analysis; and
c. database of impurities detection results/analysis

It is another object of the current invention to disclose the method as defined in any of the above, wherein the step of retrieving data from the impurities database is selected from a group consisting of pesticides, insecticides, toxins, pesticides, fungicides, Biological contaminants (fungi).

It is another object of the current invention to disclose the method as defined in any of the above, wherein the analysis comprises stages of:
a. identifying the chemical content of the identified product;
b. identifying Cannabis constituents; and
c. identifying contaminants in the sample;

It is another object of the current invention to disclose the method as defined in any of the above, wherein the sample analysis is conducted by a function selected from a group consisting of an agent, a decision tree, an algorithm and any combination thereof.

It is another object of the current invention to disclose the method as defined in any of the above, wherein the function is configured to analyze the data, the data selected from a group consisting of sample data, detection data and any combination thereof.

It is another object of the current invention to disclose the method as defined in any of the above, wherein the function is configured to detect the samples tag, classification, detection data and any combination thereof.

It is another object of the current invention to disclose the method as defined in any of the above, wherein the tag and/or classification is selected from a group consisting of chemical composition,

It is another object of the current invention to disclose the method as defined in any of the above, wherein the analysis function is determined by employing machine learning.

It is another object of the current invention to disclose the method as defined in any of the above, wherein the determining of the function comprises step of;
a. loading sample data to the the database;
b. checking the tagging of the data;
c. if sample is tagged, adding the sample for non-supervised machine learning training;
d. filtering and clustering the sample data;
e. sorting the sample data in multi-level hierarchy;
f. importing of the user medical data;
g. analyzing the relation;
h. analyzing the sample data with relevancy to the user; and
i. storing of analysis in database with recommendation.

It is another object of the current invention to disclose the method as defined in any of the above, wherein the determining of the function comprises step of;
a. loading sample data to the the database;
b. checking the tagging of the data;
c. if sample is not tagged, adding the sample for supervised machine learning training;
d. identifying features and identifications of the sample;
e. improving the analysis function;
f. storing the function in the database; and
g. comparing the new function to previous function;
h. replacing previous function, if new function is better.

It is another object of the current invention to disclose the method as defined in any of the above, wherein the method additionally comprises a step of updating a Pharmacovigilance unit 109.

It is another object of the current invention to disclose the method as defined in any of the above, wherein the step of updating the Pharmacovigilance unit comprises steps of:
a. inputting healthcare results;
b. inputting information from a healthcare professional;
c. questioning the patent.

It is another object of the current invention to disclose the method as defined in any of the above, wherein the method additionally comprises a step of updating the treatment of the patent 111.

It is another object of the current invention to disclose the method as defined in any of the above, wherein the step of updating the treatment 111 comprises steps of:
a. updating Cannabis product;
b. updating Cannabis dose;
c. updating treatment schedule;

It is the object of the current invention to disclose a system for analyzing a Tabaco product 10, comprising:
a. At least one detecting unit 11 22; the unit comprising
   i. at least one detector 92; and
   ii. at least one detection/sensor/sensing source;
   iii. a computer readable medium (CRM), the CRM configured to process the detection results;
b. a processing unit 12 23, the unit configured to read the detection results;
c. a communication unit 13 24, the unit configured to transmit the results;
d. an analysis database 15 26; and
e. an analysis unit 14 25, the unit configured to analyses the product results and compare the results to the database;
wherein the system provides a report, the report disclosing the quality of the sample.

It is another object of the current invention to disclose the system as defined in any of the above, wherein the detector 11 22 is selected from a group of technologies consisting of chemical detectors, spectroscopic detectors, electrochemical, luminescent, fluorescent, optical, polymer, hydrogels, surface acoustic wave (SAW) electronic-nose sensors, as immunological (antibody), enzyme-linked immunosorbent assay (ELISA), enzymatic biosensor microbial biosensors, fiber optic biosensors, quartz crystal microbalance (QCM) and electromagnetic QCM (EQCM).

It is another object of the current invention to disclose the system as defined in any of the above, wherein the detector is configured to detect at least one environmental condition.

It is another object of the current invention to disclose the system as defined in any of the above, wherein the conditions are selected from a group consisting of temperate and humidity.

It is another object of the current invention to disclose the system as defined in any of the above, wherein the detector is configured to analyze volatile compounds.

It is another object of the current invention to disclose the system as defined in any of the above, wherein the system additionally comprises a sample holder.

It is another object of the current invention to disclose the system as defined in any of the above, wherein the holder is configured to position a solid sample or a liquid sample.

It is another object of the current invention to disclose the system as defined in any of the above, wherein the holder additionally comprises a heating unit.

It is another object of the current invention to disclose the system as defined in any of the above, wherein the heating unit is selected from a group consisting of Infrared light, heating coil, electric element.

It is another object of the current invention to disclose the system as defined in any of the above, wherein the communication 13 unit is configured to communicate using wired or wireless technologies selected from a group consisting of, but not limited to, wireless local area networks (WLAN), Low-Power Wide-Area Networks (LPWAN), wireless wide area network (WWAN), wireless sensor networks, Bluetooth, cellular networks, and any combination of.

It is another object of the current invention to disclose the system as defined in any of the above, wherein the database 15 26 comprises at least one of the following:
1. database of Tabaco detection results/analysis;
2. database of Tabaco analysis/results;
3. database of impurities detection results/analysis.

It is another object of the current invention to disclose the system as defined in any of the above, wherein the impurities is selected from a group consisting of pesticides, insecticides, toxins, pesticides, fungicides, Biological contaminants (fungi).

It is another object of the current invention to disclose the system as defined in any of the above, wherein the system additional comprises a display for presenting the analysis results.

It is another object of the current invention to disclose the system as defined in any of the above, wherein the system is powered by an internal electricity source, the source being a battery.

It is another object of the current invention to disclose the system as defined in any of the above, wherein the analysis unit 14 25 is configured to communicate the result to another device or system.

It is another object of the current invention to disclose the system as defined in any of the above, wherein the analysis unit is configured to communicate using wired or wireless technologies selected from a group consisting of, but not limited to, wireless local area networks (WLAN), Low-Power Wide-Area Networks (LPWAN), wireless wide area network (WWAN), wireless sensor networks, Bluetooth, cellular networks, and any combination of.

It is another object of the current invention to disclose the system as defined in any of the above, wherein the analysis unit is configured to communicate using computer-mediated communication (CMC), the CMC selected from a group consisting of email, text message, and web-based application.

It is another object of the current invention to disclose the system as defined in any of the above, wherein the communication is by an application installed on a mobile device.

It is another object of the current invention to disclose the system as defined in any of the above, wherein the analysis unit is configured to mine the database for product analysis.

It is another object of the current invention to disclose the system as defined in any of the above, wherein the sample analysis is conducted by a function selected from a group consisting of an agent, a decision tree, an algorithm and any combination thereof.

It is another object of the current invention to disclose the system as defined in any of the above, wherein agent is configured to analyze the data, the data selected from a group consisting of sample data, detection data and any combination thereof.

It is another object of the current invention to disclose the system as defined in any of the above, wherein the agent is configured to detect the samples tag, classification, sample data and any combination thereof.

It is another object of the current invention to disclose the system as defined in any of the above, wherein the tag and/or classification is selected from a group consisting of chemical composition.

It is another object of the current invention to disclose the system as defined in any of the above, wherein the analysis is a function determined by employing machine learning.

It is another object of the current invention to disclose the system as defined in any of the above, wherein the system additionally comprises a reporting unit 27, the unit configured to:
a. generate an analysis report;
b. transmit analysis report to a pharmacovigilance unit 28;
c. communicate analysis results.
It is another object of the current invention to disclose the system as defined in any of the above, wherein the system additionally comprises a pharmacovigilance unit 28.
It is another object of the current invention to disclose the system as defined in any of the above, wherein the pharmacovigilance unit 28 is configured to:
a. receive health information from the patent;
b. receive health information for a healthcare person;
c. receive health information for a healthcare system;
d. receive analysis results from reporting unit;
e. communicate pharmacovigilance data to the reporting unit;

It is another object of the current invention to disclose the system as defined in any of the above, wherein the pharmacovigilance unit is configured to receive health information and to present a questionnaire to the patent or patent care giver, the questionnaire comprising questions regarding patent health, treatment effect, and additional treatments.

It is another object of the current invention to disclose the system as defined in any of the above, wherein the external person or system is a healthcare person, a patents group or a regulatory body.

It is another object of the current invention to disclose the system as defined in any of the above, wherein the system additionally comprises a product unit 21, the unit configured to input information about the product.

It is another object of the current invention to disclose the system as defined in any of the above, wherein the product information is selected from a group consisting of the product batch, manufacturing date, source, producer, manufacturer, and storage and transport conditions

It is the object of the current invention to disclose a method of analyzing a Tabaco product 100, comprising steps of
a. receiving a Tabaco product 101;
b. obtaining a Tabaco sample 102;
c. providing an detection unit;
d. analyzing the sample with the detection unit 103;
e. processing results 104;
f. transferring results to the analysis unit 105;
g. analyzing results 106;
h. retrieving data from a database 107; and
i. generating a report of the analysis 108;
   wherein the report comprises an analysis of the Tabaco product.

It is another object of the current invention to disclose the method as defined in any of the above, wherein the method additionally comprises a step of heating the sample.

It is another object of the current invention to disclose the method as defined in any of the above, wherein the step of heating the sample is conducted by using a technology selected from a group consisting of Infrared light, heating coil, and electric heater.

It is another object of the current invention to disclose the method as defined in any of the above, wherein the method additionally comprises a step of evaporating volatile compounds of the sample.

It is another object of the current invention to disclose the method as defined in any of the above, wherein the step of detecting the composition of the sample is conducted by a technology selected from a group consisting of chemical detectors, spectroscopic detectors, electrochemical, luminescent, fluorescent, optical, polymer, hydrogels, surface acoustic wave (SAW) electronic-nose sensors, as immunological (antibody), enzyme-linked immunosorbent assay (ELISA), enzymatic biosensor microbial biosensors and fiber optic biosensors

It is another object of the current invention to disclose the method as defined in any of the above, wherein the method additionally comprises a step of retrieving data from a database 107, the database comprising:
a. database of Tabaco detection results/analysis;
b. database of Tabaco detection results/analysis; and
c. database of impurities detection results/analysis
It is another object of the current invention to disclose the method as defined in any of the above, wherein the step of retrieving data from the impurities database is selected from a group consisting of pesticides, insecticides, toxins, pesticides, fungicides, Biological contaminants (fungi).

It is another object of the current invention to disclose the method as defined in any of the above, wherein the analysis comprises stages of:
a. identifying the chemical content of the identified product;
b. identifying constituents; and
c. identifying contaminants in the sample;
It is another object of the current invention to disclose the method as defined in any of the above, wherein the sample analysis is conducted by a function selected from a group consisting of an agent, a decision tree, an algorithm and any combination thereof.

It is another object of the current invention to disclose the method as defined in any of the above, wherein the agent is configured to analyze the data, the data selected from a group consisting of sample data, detection data and any combination thereof.

It is another object of the current invention to disclose the method as defined in any of the above, wherein the agent is configured to detect the samples tag, classification, sample data and any combination thereof.

It is another object of the current invention to disclose the method as defined in any of the above, wherein the tagging/classification is selected from a group consisting of chemical composition,

It is another object of the current invention to disclose the method as defined in any of the above, wherein the analysis function is determined by employing machine learning.

It is another object of the current invention to disclose the method as defined in any of the above, wherein the determining of the function comprises step of;
a. loading sample data to the database;
b. checking the tagging of the data;
c. if sample is tagged, adding the sample for non-supervised machine learning training;
d. filtering and clustering the sample data;
e. sorting the sample data in multi-level hierarchy;
f. imploring of the user medical data;
g. analyzing the relation;
h. analyzing the sample data with relevancy to the user; and
i. storing of analysis in database with recommendation.

It is another object of the current invention to disclose the method as defined in any of the above, wherein the determining of the function comprises step of;
a. loading sample data to the database;
b. checking the tagging of the data;
c. if sample is not tagged, adding the sample for supervised machine learning training;
d. identifying features and identifications of the sample;
e improving the analysis function;
f. storing the function in the database; and
g. comparing the new function to previous function;
h. replacing previous function, if new function is better.

It is another object of the current invention to disclose the method as defined in any of the above, wherein the method additionally comprises a step of updating a Pharmacovigilance unit 109.

It is another object of the current invention to disclose the method as defined in any of the above, wherein the step of updating the Pharmacovigilance unit comprises steps of:
a. inputting healthcare results;
b. inputting information from a healthcare professional;
c. questioning the patent.

It is another object of the current invention to disclose the method as defined in any of the above, wherein the method additionally comprises a step of updating the use of the patent 111.

It is another object of the current invention to disclose the method as defined in any of the above, wherein the step of updating the treatment 111 comprises steps of:
a. updating Tabaco product;
b. updating Tabaco dose;
c. updating use schedule;

### DETAILED DESCRIPTION OF THE PREFERRED EMBODIMENTS

The accompanying drawings, which are included to provide a further understanding of the invention and are incorporated in and constitute a part of this specification, illustrate embodiments of the invention and together with the description serve to explain the principles of the invention wherein:

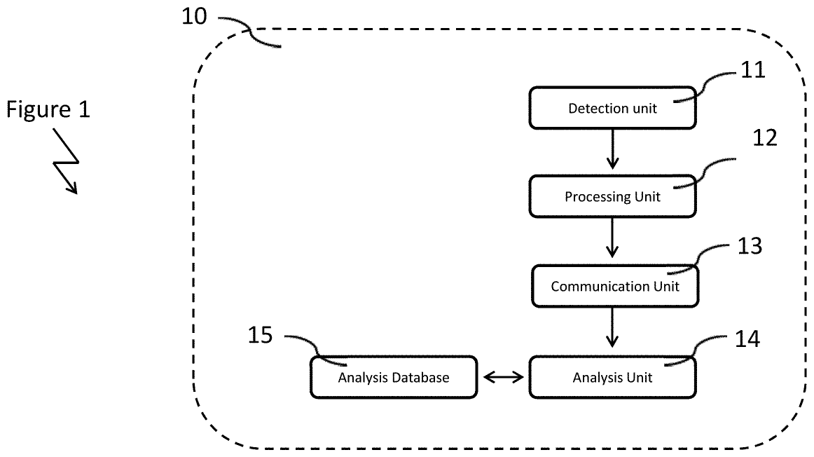

FIG. 1—discloses a schematic of a purposes embodiment of a system for the analysis of a Cannabis product 10

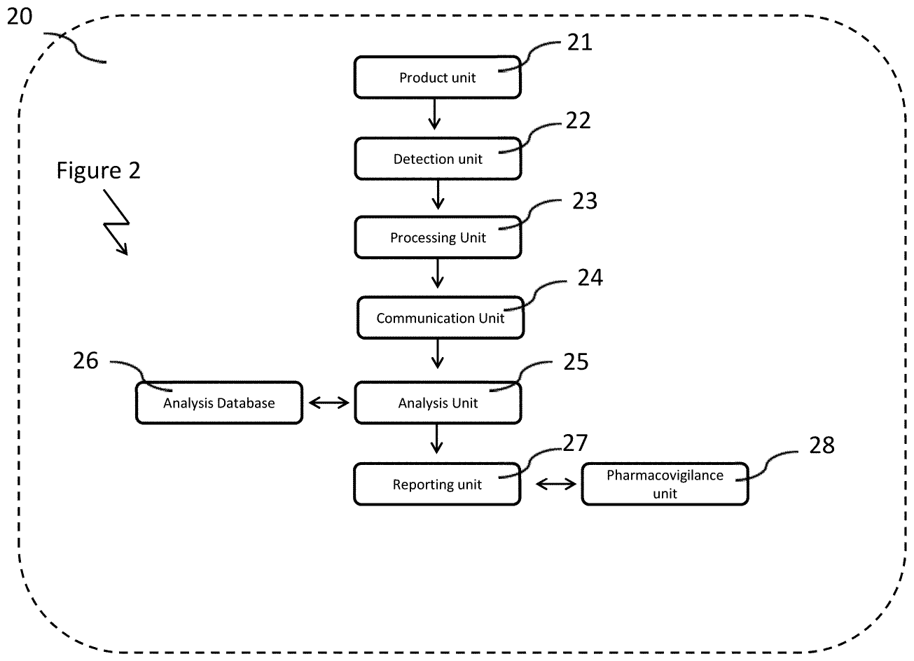

FIG. 2—discloses a schematic of a second purposes embodiment of a system for the analysis of a Cannabis product and the health effect thereof 20

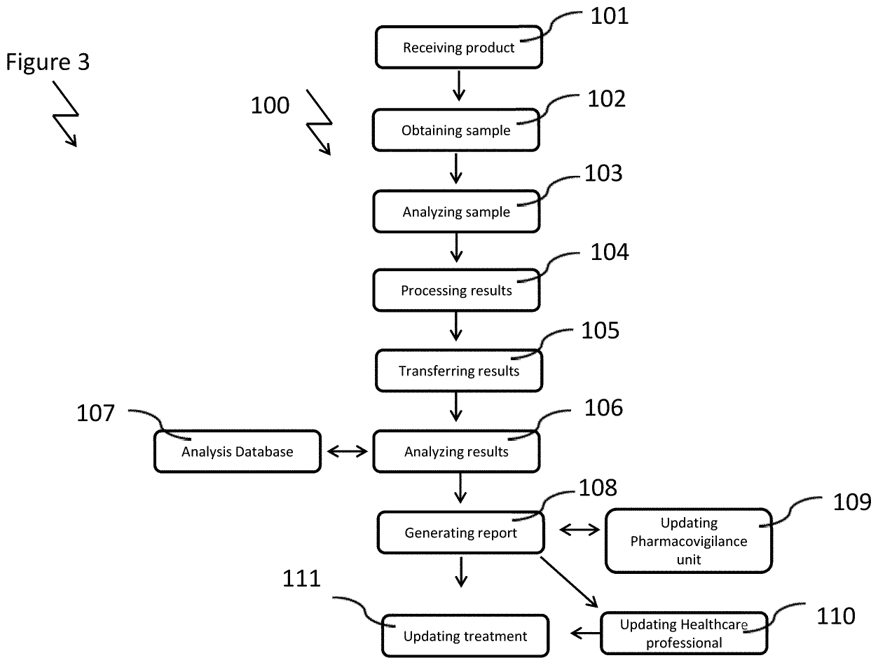

FIG. 3—discloses a schematic of a purposes method for the analysis of a Cannabis product 100

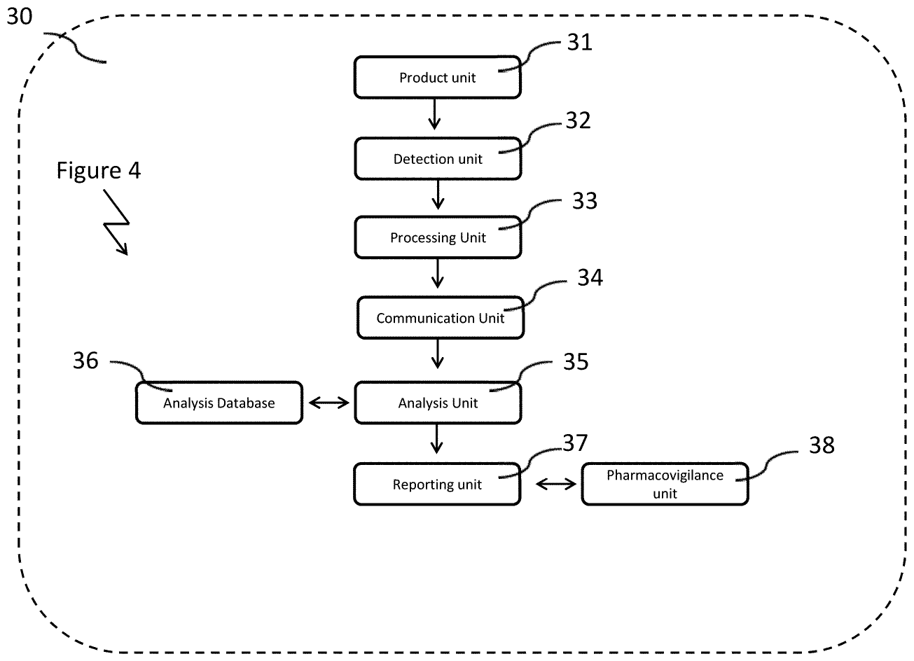

FIG. 4—discloses a schematic of a second purposes embodiment of a system for the analysis of a tobacco product and the health effect thereof 30

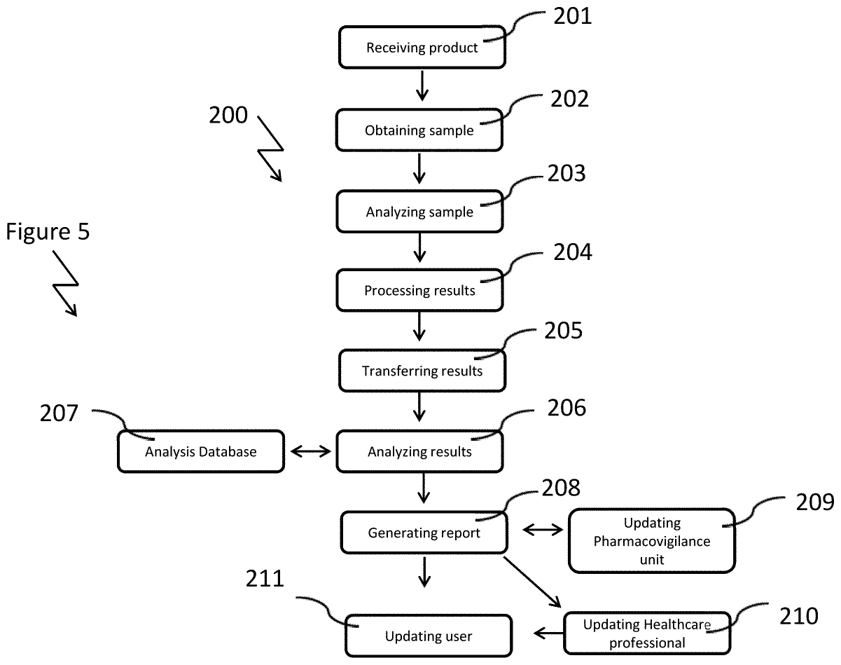

FIG. 5—discloses a schematic of a purposes method for the analysis of a tobacco product 200

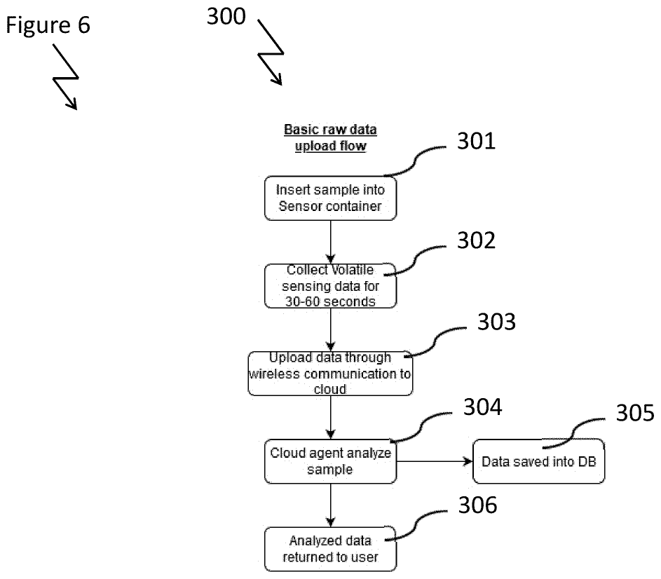

FIG. 6—discloses the analysis work flow of the present invention 300

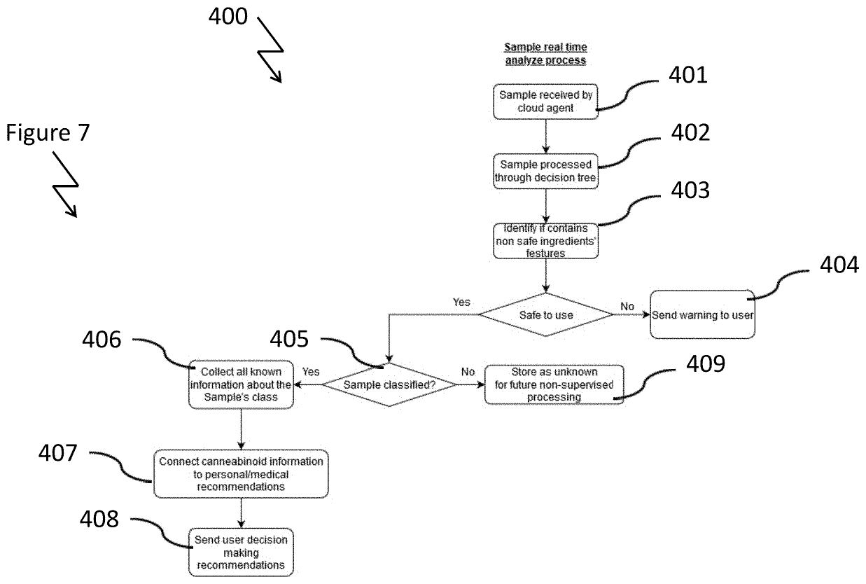

FIG. 7—discloses the analysis of the presenting invention 400

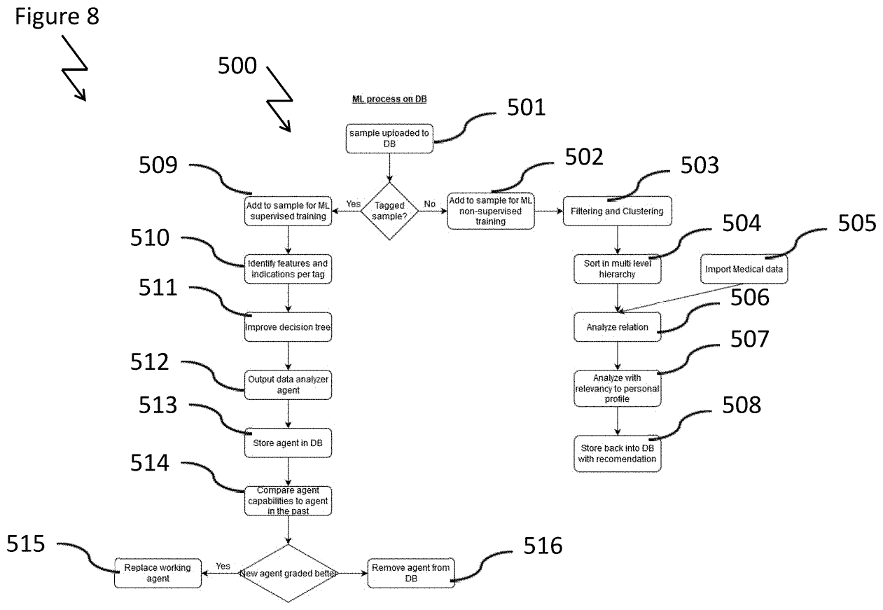

FIG. 8—discloses the work flow for the machine learning of the present invention 500

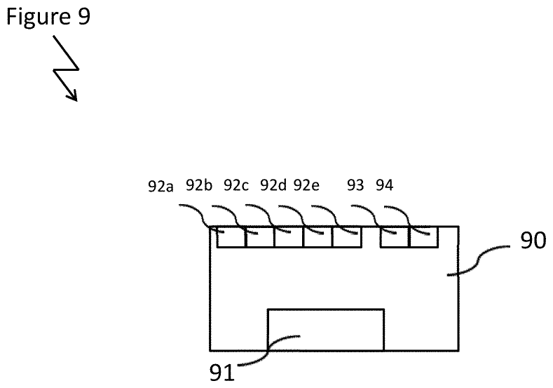

FIG. 9—discloses the schematic of the depiction unit

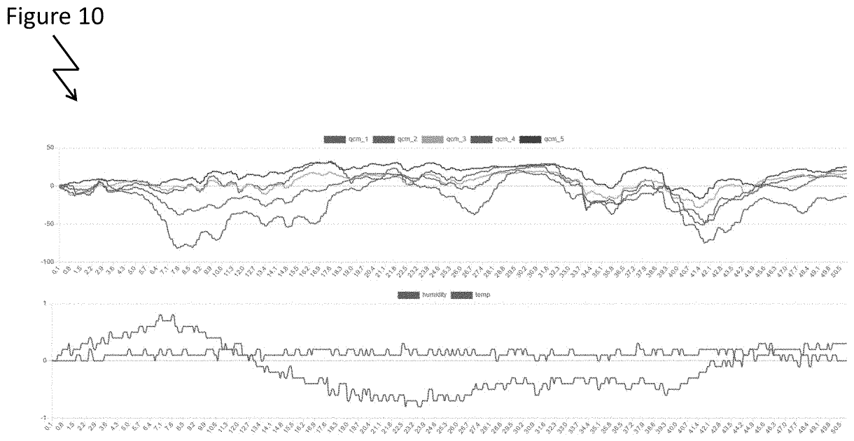

FIG. 10—presenting the analysis of a sample by the present invention

### DETAILED DESCRIPTION OF THE PREFERED EMBODIMENT

The following description is provided, alongside all chapters of the present invention, so as to enable any person skilled in the art to make use of the invention and sets forth the best modes contemplated by the inventor of carrying out this invention. Various modifications, however, are adapted to remain apparent to those skilled in the art, since the generic principles of the present invention have been defined specifically to provide compositions and methods.

In this application the term computer system, a processor, or a computer-readable medium (CRM) should be understood to also constituted a software product installed on a “server” system, e.g., “cloud computer”, or may be “client computer”, e.g. API in client's personal communication device, such as a mobile phone, or various modules of the computer system may be distributed between the server and client computers (i.e. distributed software). The software may also be installed on a processor build into the analysis devise.

In this application, the term detection unit refers to a unit comprising a sensor (single-element or multi-element sensor) implementing any known suitable sensing technique(s) for sensing/detecting chemical and/or biochemical and/or physical parameters characterizing media in the vicinity of the sensor (as well as dynamic changes of such parameters) and translating them into “readable” signals (e.g. electrical signals). Chemical and biochemical sensing provide for determining material-related parameter(s) of the media (concentration/amount of chemical and/or biological quantity); while physical sensing provides for determining parameter(s) indicative of physical interaction with various materials, as well as such parameters as pressure, displacement, temperature within the media. The detection unit further comprises a processor to process the detection results for further analysis.

In this application the term function refers to the object configured to conduct analysis of the analysis data, such as an agent, decision tree, algorithm, classification, filtering and other known analysis and pattern recondition methods.

In this application the term agent refers to—the run time object in the cloud which analyze the data through its algorithms (Classifier). In the present invention the front end of the system receives the sample data from the remote device and analyzes it.

In this application the term decision tree (DT) refers to an example of one of the possible classifiers (Classifier Method). In the present invention the DT could be an algorithm which analyzed olfactory data and its output is the classification of the product

In this application the term tag refers to—The target classes the classifier is aim to divide the samples into, as they are attached to the samples used for training. In the present invention Product ID, Group of products, pesticide, Mold or any other specific data on the sample could be considered as a tag.

In this application the term classification refers to—The sorting of the sample, into classes. In the present invention, sorting the analyzed sample by the device, to a class, which the system can provide useful information about it for the consumer.

In this application the term supervised/unsupervised training refers to the training process, which may be based on classified data or unclassified data. When the training is based on unclassified data it is considered to be unsupervised. When the training is based on classified data it is considered to be supervised. In the present invention, known samples collected in controlled environments (i.e. in a laboratory) will be used for supervised training. Data collected from unknown samples (i.e. from the public) will be used for unsupervised training.

In this application, the term filtering refers to the sorting of samples that can be classified and those which cannot be. In the present invention contaminated samples (i.e. which contains smells from the surrounding or in contaminated chamber), will be filtered out from training.

In this application, the clustering refers to—An unsupervised classification. In the present invention, unknown samples (such as data collected by the public) will be classified into unknown classes, to be used by analytical techniques (such as data mining) to identify correlations or links between different samples (known and unknown).

### Embodiments

It is the scope of the application to describe a system for the analysis of Cannabis products 10. The system is schematically described in FIG. 1. The system comprises a detection unit 11. The detection unit comprises a sensor source and a sensor detector. The sensing can be based on numerous technologies, such as chemical detectors, spectroscopic detectors, electrochemical, luminescent, fluorescent, optical, polymer, hydrogels, surface acoustic wave (SAW) electronic-nose sensors, as immunological (antibody), enzyme-linked immunosorbent assay (ELISA), enzymatic biosensor microbial biosensors and fiber optic biosensors.

In some embodiments the detection unit has a sample holder. The sample holder can hold a solid (such as Cannabis plant matter/leaves) or a liquid (such as Cannabis oil). The sample holder can regulate the temperature sample to achieve more reproducible analysis. The sample holder can heat the sample to evaporate the volatile compounds to enable selective analysis.

In some embodiments the detection unit comprises a computer readable medium (CRM) configured to read the analysis and convert it to a data version suitable for further processing.

In some embodiment the device comprises a processing unit 12, configured to process the analysis results. The analysis is converted to a readable format for further analysis or to be communicated to another computer. The system comprises a communication unit 13 that can communicate the analysis results with a database 15. The database can be situated on the devise, on a mobile devise or on a central computer server (on the cloud). The communication unit can communicate using wireless local area networks (WLAN), Low-Power Wide-Area Networks (LPWAN), wireless wide area network (WWAN), wireless sensor networks, Bluetooth, cellular networks.

The database comprises the analysis of compounds that are in the sample. These compounds can be constituents of the Cannabis (such as cannabinoids) or other added chemicals (such as pesticides or insecticides). The system comprises an analysis unit 14 that is configured to analyses the product results using the database. The analysis unit generates a report, detailing the chemical composition of the sample. In some embodiments the report is displayed on the device or the report can be sent to another unit. This unit can be an application on a mobile device.

FIG. 2 schematically describes a system that can track a Cannabis product and treatment 20: from purchase through analysis to treatment. The system comprises a product unit 21 for imputing information regarding the product. This information can include the product batch, manufacturing date, source, producer, manufacturer, and storage and transport conditions. This information could be imputed by the user via a keyboard or an optical technology such as a barcode reader linked to a data base. The barcode could be a 2 or 3-dementional code.

The system comprises a detection unit 22. The detection unit comprises a sensor source and a sensor detector. The sensing can be based on numerous technologies, such as chemical detectors, spectroscopic detectors, electrochemical, luminescent, fluorescent, optical, polymer, hydrogels, surface acoustic wave (SAW) electronic-nose sensors, as immunological (antibody), enzyme-linked immunosorbent assay (ELISA), enzymatic biosensor microbial biosensors, fiber optic biosensors, quartz crystal microbalance (QCM) and electromagnetic QCM (EQCM).

When the detector is a QCM, the mass the change in frequency of a quartz crystal resonator is disturbed by the interaction of the sample with the surface of the acoustic resonator. The surface of each of the QCMs would be different. The interaction(s) of the surface with the chemical components of the sample could be characterized as some combination of specific, selective, non-selective and/or non-specific interaction(s). In some embodiments, the signals(s) generated by the QCM(s) is specific to the chemical composition of the sample. In some embodiments, the combination of signals from the various QCM(s) is specific to the chemical composition of the sample.

In some embodiments, the detection unit comprises a both selective/specific and non-selective/specific sensors. In some embodiments, the detection unit further comprises environmental sensors, such as humidity and temperature.

In some embodiments the detection unit has a sample holder. The sample holder can hold a solid (such as Cannabis leaves) or a liquid (such as Cannabis oil). The sample holder can regulate the temperature sample to achieve more reproducible analysis. The sample holder can heat the sample to evaporate the volatile compounds to enable selective analysis.

In some embodiments the detection unit comprises a computer readable medium (CRM) configured to read the analysis and convert it to a data version suitable for further processing.

In some embodiment the device comprises a processing unit 23, configured to process the analysis results. The analysis is converted to a readable format for further analysis or to be communicated to another computer. The system comprises a communication unit 24 that can communicate the analysis results with a database 26. The database can be situated on the devise, on a mobile devise or on a central computer server (on the cloud). The communication unit can communicate using wireless local area networks (WLAN), Low-Power Wide-Area Networks (LPWAN), wireless wide area network (WWAN), wireless sensor networks, Bluetooth, cellular networks.

The database comprises the analysis of compounds that are in the sample. These compounds can be constituents of the Cannabis (such as cannabinoids) or other added chemicals (such as pesticides or insecticides). The system comprises an analysis unit 25 that is configured to analyses the product results using the database. In some embodiments, the analysis unit is configured to analysis the specific, selective, non-selective and/or non-specific readings/signals generated by the detection units. In some embodiments, the analysis unit is configured to analyze signals(s) generated by the sensors (such as QCM(s) based sensors) is specific to the chemical composition of the sample. In some embodiments, the combination of signals from the various sensors(s) is specific to the chemical composition of the sample. In some embodiments, the analysis unit is configured to analyze environmental data, such as humidity and temperature.

In some embodiments the system comprises a reporting unit 27 that generates a report, detailing the chemical composition of the sample. In some embodiments the report is displayed on the device or the report can be sent to another unit. This unit can be an application on a mobile device. In some embodiments the analysis is conveyed to another person, such as a healthcare professional.

In some embodiments, system comprises a pharmacovigilance unit 28 that is configured to track the health of the patent and the status of the product analysis. This can be tracked by inputting healthcare results, information from a healthcare professional or by presenting the patent with a questionnaire. The health status of the patent is stored to enable better understanding of the treatments effectiveness. The pharmacovigilance unit can receive analysis date from the analysis unit to combine with the

[symbol]

FIG. 3 describes a method for the analysis of a Cannabis product 100. The product can be Cannabis or a Cannabis product. The product could be an extract, oil or a solution comprising cannabinoids. The product information is entered 101 to the product unit. This information can be entered manually, by means of a key board, or automatically, such as by reading a bar-code. This information may include the product batch, manufacturing date, source, producer, manufacturer, and storage and transport conditions. The user may also add additional information, such as the status of the package. A sample of the product is taken 102 and positioned for detection. The detector is activated and the sample is analyzed 103. The analysis may detect the composition of the sample including cannabinoids and contaminates, such as pesticides. If the sample is of a cannabinoid formulation the detector may also analyses the additional components of the formulation, such as solvents. The analysis results are processed 104 and transferred to the analysis unit 105. The analysis retrieves data from the analysis database 107 and analysis the sample 106. A report describing the chemical composition of the sample is generated 108.

In some embodiments, the report is sent to a healthcare professional for further treatment 110.

In some embodiments a Pharmacovigilance unit is updated 109 with a report of the patents health state. The patents health status is recorded by a healthcare professional or by generating a questionnaire for the patent.

Based on the report and the healthcare professional's opinion, the treatment may be updated 111 or changed to produce a better result.

FIG. 4 schematically describes a system that can track a tobacco product and treatment 30: from purchase through analysis to treatment. The system comprises a product unit 31 for imputing information regarding the product. This information can include the product batch, manufacturing date, source, producer, manufacturer, and storage and transport conditions. This information could be imputed by the user via a keyboard or an optical technology such as a barcode reader linked to a data base. The barcode could be a 2 or 3-dementional code.

The system comprises a detection unit 32. The detection unit comprises a sensor source and a sensor detector. The sensing can be based on numerous technologies, such as chemical detectors, spectroscopic detectors, electrochemical, luminescent, fluorescent, optical, polymer, hydrogels, surface acoustic wave (SAW) electronic-nose sensors, as immunological (antibody), enzyme-linked immunosorbent assay (ELISA), enzymatic biosensor microbial biosensors, fiber optic biosensors, quartz crystal microbalance (QCM) and electromagnetic QCM (EQCM).

When the detector is a QCM, the mass the change in frequency of a quartz crystal resonator is disturbed by the interaction of the sample with the surface of the acoustic resonator. The surface of each of the QCMs would be different. The interaction(s) of the surface with the chemical components of the sample could be characterized as some combination of specific, selective, non-selective and/or non-specific interaction(s). In some embodiments, the signals(s) generated by the QCM(s) is specific to the chemical composition of the sample. In some embodiments, the combination of signals from the various QCM(s) is specific to the chemical composition of the sample.

In some embodiments, the detection unit comprises a both selective/specific and non-selective/specific sensors. In some embodiments, the detection unit further comprises environmental sensors, such as humidity and temperature.

In some embodiments the detection unit has a sample holder. The sample holder can hold a solid (such as tobacco leaves) or a liquid (such as tobacco solution). The sample holder can regulate the temperature sample to achieve more reproducible analysis. The sample holder can heat the sample to evaporate the volatile compounds to enable selective analysis.

In some embodiments the detection unit comprises a computer readable medium (CRM) configured to read the analysis and convert it to a data version suitable for further processing.

In some embodiment the device comprises a processing unit 33, configured to process the analysis results. The analysis is converted to a readable format for further analysis or to be communicated to another computer. The system comprises a communication unit 34 that can communicate the analysis results with a database 36. The database can be situated on the devise, on a mobile devise or on a central computer server (on the cloud). The communication unit can communicate using wireless local area networks (WLAN), Low-Power Wide-Area Networks (LPWAN), wireless wide area network (WWAN), wireless sensor networks, Bluetooth, cellular networks.

The database comprises the analysis of compounds that are in the sample. These compounds can be constituents of the tobacco (such as nicotine) or other added chemicals (such as pesticides or insecticides). The system comprises an analysis unit 35 that is configured to analyses the product results using the database. In some embodiments, the analysis unit is configured to analysis the specific, selective, non-selective and/or non-specific readings/signals generated by the detection units. In some embodiments, the analysis unit is configured to analyze signals(s) generated by the sensors (such as QCM(s) based sensors) is specific to the chemical composition of the sample. In some embodiments, the combination of signals from the various sensors(s) is specific to the chemical composition of the sample. In some embodiments, the analysis unit is configured to analyze environmental data, such as humidity and temperature.

In some embodiments the system comprises a reporting unit 37 that generates a report, detailing the chemical composition of the sample. In some embodiments the report is displayed on the device or the report can be sent to another unit. This unit can be an application on a mobile device. In some embodiments the analysis is conveyed to another person, such as a healthcare professional.

In some embodiments, system comprises a pharmacovigilance unit 38 that is configured to track the health of the patent and the status of the product analysis. This can be tracked by inputting healthcare results, information from a healthcare professional or by presenting the patent with a questionnaire. The health status of the patent is stored to enable better understanding of the treatments effectiveness. The pharmacovigilance unit can receive analysis date from the analysis unit to combine with the

[symbol]

FIG. 5 describes a method for the analysis of a tobacco product 200. The product can be tobacco leaves or a tobacco product. The product could be an extract, oil or a solution comprising tobacco extracts. The product information is entered 201 to the product unit. This information can be entered manually, by means of a key board, or automatically, such as by reading a bar-code. This information may include the product batch, manufacturing date, source, producer, manufacturer, and storage and transport conditions. The user may also add additional information, such as the status of the package. A sample of the product is taken 202 and positioned for detection. The detector is activated and the sample is analyzed 203. The analysis may detect the composition of the sample including nicotine and contaminates, such as pesticides. If the sample is of a formulation the detector may also analyses the additional components of the formulation, such as solvents. The analysis results are processed 204 and transferred to the analysis unit 205. The analysis retrieves data from the analysis database 207 and analysis the sample 206. A report describing the chemical composition of the sample is generated 208.

In some embodiments, the report is sent to a healthcare professional for further treatment 210.

In some embodiments a Pharmacovigilance unit is updated 209 with a report of the user's health state. The user's health status is recorded by a healthcare professional or by generating a questionnaire for the patent.

Based on the report and the healthcare professional's opinion, a recommendation to change the tobacco user's consumption may be made.

Reference is made to FIG. 6, presenting the work flow 300 for a method of analyzing a sample, comprising:
1. Loading a sample into the container 301. In some embodiments, the sample is loaded in to a sample holder.
2. Collecting the sensing data of the volatile compounds 302, mainly for 30-60 seconds. In some embodiments the sample is heated to a controlled temperature, to regulate the evaporation of the chemical contents.
3. Uploading the sensing data to a cloud-based database 303, commonly though wireless communications technologies.
4. Analyzing the data, by a cloud-based database 304.
5. Saving the data and results to the database 305.
6. Presenting the analysis results to the user 306.

Reference is made to FIG. 7, presenting the real-time sample analysis by a cloud-based agent 400, comprising steps of:
1. Receiving the sample data 401 from the detection unit. The analysis data may be generated from various types of sensor or sensor interfaces. In some embodiments the analysis data is created detection-by-time graph.
2. Processing the sample data through the decision three 402. In some embodiments, the decision tree can be based on various segments of the analysis, such as the height, shape, rate or other shapes of the graph at specific points along the analysis, in a time and/or temperature dependent manor
3. Identifying unsafe ingredients and contaminants in the sample 403;
4. IF the sample is safe to use—sending a warning to the user 404;
5. IF sample is safe to use—checking sample classification 405;
   5.1. IF sample is classified—collecting all known and related data regarding the sample class 406;
      5.1.1. Connecting cannabinoid information to personal/medical information 407;
      5.1.2. Sending the decision making recommendations to the user 408;
   5.2. IF sample is not classified—storing sample data for future non-supervised processing 409.

Reference is made to FIG. 8, presenting a work flow 500 for a method of machine learning (ML) process in the database (DB):
1. Loading sample data to the DB;
2. Checking the tagging of the data;
3. IF sample is tagged—adding to sample for ML non-supervised training 502;
   3.1. Filtering and clustering 503.
   3.2. Sorting in multi-level hierarchy 504.
   3.3. Imploring of medical data 505;
   3.4. Analyzing relation 506.
   3.5. Analyzing with relevancy to personal profile 507; and
   3.6. Storing of analysis in DB with recommendation 508.
4. IF sample is not tagged—adding to sample for ML supervised training 509;
   4.1. Identify features and identifications per tag 510;
   4.2. Improve decision tree 511;
   4.3. Output data analyzer agent 512;
   4.4. Store agent in DB 513; and
   4.5. Compare agent capabilities to agent in past 514;
      4.5.1. IF new agent graded better—replace working agent 515;
      4.5.2. IF new agent does not graded better—remove agent from DB 515;

Reference is made to FIG. 9, presenting a schematic depiction of the detection unit 11 21, having a detection chamber 90, comprising a sample holder 91. The sample holder is configured to regulate the temperature of the sample. The detection chamber comprises a plurality of quartz crystal microbalance (QCM) based sensors 92a-e, each sensor comprising a different interface. The environmental conditions of the detection chamber are monitored by temperate 93 and humidity sensors 94.

Reference is made to FIG. 10, presenting the real-time analysis over 50.5 seconds, of a Cannabis-based sample by the detection unit of FIG. 9, comprising 5 QCM-based sensors, a temperature sensor and a humidity sensor.

The specific example analyzed is known to contain no harmful agents (such as molds or pesticides). Analysis of the example by the system would not generate a warning to the user, that the sample is not safe to use 404.

1.-101. (canceled)

102. A system for analyzing a Cannabis product 10, comprising:
a. At least one detecting unit 11 22; said unit comprising
i. at least one detector 92;
ii. at least one detection/sensor/sensing source; and
iii. a computer readable medium (CRM), said CRM configured to process the detection results;
b. a processing unit 12 23, said unit configured to read the detection results;
c. a communication unit 13 24, said unit configured to transmit said results;
d. an analysis database 15 26; and
e. an analysis unit 14 25, said unit configured to analyses said product results and compare said results to said database;
wherein said system provides a report, said report disclosing the quality of said sample.

103. The system of claim 102, wherein said detector 11 22 is selected from a group of technologies consisting of chemical detectors, spectroscopic detectors, electrochemical, luminescent, fluorescent, optical, polymer, hydrogels, surface acoustic wave (SAW) electronic-nose sensors, as immunological (antibody), enzyme-linked immunosorbent assay (ELISA), enzymatic biosensor microbial biosensors, fiber optic biosensors, quartz crystal microbalance (QCM) and electromagnetic QCM (EQCM).

104. The system of claim 102, wherein said detector is configured to detect at least one environmental condition.

105. The system of claim 104, wherein said conditions are selected from a group consisting of temperate and humidity.

106. The system of claim 102, wherein said detector is configured to analyze volatile compounds.

107. The system of claim 102, wherein said system additionally comprises a sample holder.

108. A method of analyzing a Cannabis product 100, comprising steps of
a. receiving a Cannabis product 101;
b. obtaining a Cannabis sample 102;
c. providing an detection unit;
d. analyzing said sample with the detection unit 103;
e. processing results 104;
f. transferring results to the analysis unit 105;
g. analyzing results 106;
h. retrieving data from a database 107; and
i. generating a report of said analysis 108;
wherein said report comprises an analysis of said Cannabis product.

109. The method of claim 108, wherein said method additionally comprises a step of heating said sample.

110. The method of claim 108, wherein said step of heating said sample is conducted by using a technology selected from a group consisting of Infrared light, heating coil, and electric heater.

111. The method of claim 108, wherein said method additionally comprises a step of evaporating volatile compounds of said sample.

112. The method of claim 108, wherein said step of detecting the composition of said sample is conducted by a technology selected from a group consisting of chemical detectors,
spectroscopic detectors, electrochemical, luminescent, fluorescent, optical, polymer, hydrogels, surface acoustic wave (SAW) electronic-nose sensors, as immunological (antibody), enzyme-linked immunosorbent assay (ELISA), enzymatic biosensor microbial biosensors and fiber optic biosensors.

113. The method of claim 108, wherein said method additionally comprises a step of retrieving data from a database 107, said database comprising:
a. database of medicinal Cannabis detection results/analysis;
b. database of cannabinoid detection results/analysis; and
c. database of impurities detection results/analysis

114. The method of claim 113, wherein said step of retrieving data from said impurities database is selected from a group consisting of pesticides, insecticides, toxins, pesticides, fungicides, Biological contaminants (fungi).

115. A system for analyzing a Tabaco product 10, comprising:
a. At least one detecting unit 11 22; said unit comprising
i. at least one detector 92; and
ii. at least one detection/sensor/sensing source;
iii. a computer readable medium (CRM), said CRM configured to process the detection results;
b. a processing unit 12 23, said unit configured to read the detection results;
c. a communication unit 13 24, said unit configured to transmit said results;
d. an analysis database 15 26; and
e. an analysis unit 14 25, said unit configured to analyses said product results and compare said results to said database;
wherein said system provides a report, said report disclosing the quality of said sample.

116. The system of claim 115, wherein said detector 11 22 is selected from a group of technologies consisting of chemical detectors, spectroscopic detectors, electrochemical, luminescent, fluorescent, optical, polymer, hydrogels, surface acoustic wave (SAW) electronic-nose sensors, as immunological (antibody), enzyme-linked immunosorbent assay (ELISA), enzymatic biosensor microbial biosensors, fiber optic biosensors, quartz crystal microbalance (QCM) and electromagnetic QCM (EQCM).

117. The system of claim 115, wherein said detector is configured to detect at least one environmental condition.

118. The system of claim 117, wherein said conditions are selected from a group consisting of temperate and humidity.

119. The system of claim 115, wherein said detector is configured to analyze volatile compounds.

120. The system of claim 115, wherein said system additionally comprises a sample holder.

121. The system of claim 120, wherein said holder is configured to position a solid sample or a liquid sample.
